(19) United States
(12) Patent Application Publication
(10) Pub. No.: US 2002/0142361 A1
(43) Pub. Date: Oct. 3, 2002

(54) Biodetection method

(76) Inventor: Michael R. Emmert-Buck, Silver Spring, MD (US)

Correspondence Address: Peter A. Borsari, Esq., Borsari & Associates, P.C., Suite 206, 2001 Jefferson Davis Highway, Arlington, VA, 22202, US

(21) Appl. No.: 10/102,859

(22) Filed: Mar. 22, 2002

Related U.S. Application Data
(60) Provisional application No. 60/278,745, filed on Mar. 27, 2001.

(51) Int. Cl. G01N 33/53; G01N 33/537; G01N 33/543

(52) U.S. Cl. 435/7.92

(57) ABSTRACT

A new and useful biodetection method for identifying a target biomolecule within a sample is provided which permits the accurate measurement of DNA, mRNA or protein in a complex biological sample. In particular, the inventive biodetection method provides a new method for identifying a target biomolecule in a sample in which the target biomolecule is present in low abundance relative to one or more non-target biomolecules, or when the target biomolecule and a non-target biomolecule have a shared epitope or nucleic acid sequence. The biodetection method utilizes a combination of at least one target probe and a competitor molecule in order to identify a target biomolecule in a sample. When the sample contains the target biomolecule and a non-target biomolecule having a shared epitope or nucleic acid sequence, two target probes are utilized in combination with a competitor molecule. A detectable signal is produced when the at least one target probe is in high concentration relative to the competitor molecule, thereby identifying the target biomolecule. If the competitor molecule is in high concentration in the vicinity of the at least one target probe, then a detectable signal is not produced.

### RELATED APPLICATION INFORMATION

This application claims priority from U.S. Provisional Application No. 60/278,745, filed in the United States Patent & Trademark Office on Mar. 27, 2001 in the name of Michael R. Emmert-Buck.

### FIELD OF INVENTION

The present invention relates to a biodetection method for identifying a target biomolecule within a sample. More specifically, the present invention relates to a biodetection method for identifying a target biomolecule in a sample in which the target biomolecule is present in low abundance relative to one or more non-target biomolecules, or when the target biomolecule and a non-target biomolecule have a shared epitope or similar nucleic acid sequence. The inventive biodetection method utilizes a combination of at least one target probe and a competitor molecule in order to identify a target biomolecule in a sample. A detectable signal is produced when the at least one target probe is in high concentration relative to the competitor molecule, thereby identifying the target biomolecule. If the competitor molecule is in high concentration in the vicinity of the at least one target probe, a detectable signal is not produced.

### BACKGROUND OF THE INVENTION

The completion of the Human Genome Project has increased the number of known genes from approximately 10% to nearly 100% in a short period of time. This extraordinary accomplishment has resulted in the determination of a large number of new target biomolecules that investigators and clinicians will want to study in various biological systems. An analysis of human mRNA and proteomic expression databases by the present inventor indicates that the gene products of the 10% of genes previously known are highly abundant. These transcripts and proteins have been identified, sequenced and characterized in traditional biochemical and molecular biology studies over the past several decades because they could be readily analyzed. However, the majority of new genes identified by the Human Genome Product are expressed at moderate or low abundance levels and will therefore be more technically challenging to measure and study.

Analysis of individual biomolecules plays a critical role in medical research and diagnostic clinical testing. For example, an antibody (ab) that is specific for a cellular protein permits precise measurement of the abundance level of that protein in a complex biological sample. To date, the use of molecular detection methods has been largely restricted to biomolecules that are highly abundant. Measurement of less abundant molecules has been challenging due to probe cross-reaction with non-target molecules. Therefore, as the majority of new genes identified by the Human Genome Project are expressed at moderate or low abundance levels, a new biodetection method is needed to measure these gene products.

The use of blotting techniques has been widely used to identify a target biomolecule in a mixture of biomolecules, after separation on a polyacrylamide gel. A standard immunoblot is the Western Blot (protein blotting). Other blotting methods include the Southern Blot, (DNA blotting) and the Northern Blot (RNA blotting). However, there are at least two scenarios under which blotting techniques can fail. In the first scenario, if the target biomolecule is present in low abundance relative to one or more non-target biomolecules in the sample being tested, even a relatively weak interaction between a target probe and a highly abundant non-target biomolecule will generate a significant artifactual signal relative to the signal produced by the target biomolecule. This artifactual signal will produce uncertainty as to which band on the blot represents the target biomolecule and which band is due to target probe cross-reaction with a non-target biomolecule.

In the second scenario, the target probe specifically cross-reacts with a biomolecule other than the target biomolecule due to a shared epitope between the target and non-target biomolecule. This scenario can be problematic for both target and non-target biomolecules of any abundance level. However, the problem becomes particularly challenging when the target biomolecule is in low abundance and/or the non-target biomolecule is in high abundance.

A need exists for a biodetection method that can identify target biomolecules present in a sample in low abundance, relative to the abundance of non-target biomolecules in the sample. A need also exists for a biodetection method which can identify a target biomolecule in a sample having a mixture of target and non-target biomolecules having a shared epitope, regardless of the abundance level of the target and non-target biomolecules. Such a biodetection method should be capable of enabling accurate measurement of a biomolecule in a sample, notwithstanding the abundance level of the target biomolecule. Such a method also should eliminate artifactual signals produced by cross-reaction of the target probe with the non-target biomolecule. In addition, such a method should be capable of being used with any standard blotting technique, include the immunoblot (Western blot), Southern blot and Northern blot. Moreover, such a method should be capable of detecting biomolecules in complex mixtures including, for example, histological tissue sections, cell lysates and body fluids.

### SUMMARY OF THE INVENTION

Accordingly, it is an object of the present invention to provide a biodetection method that can identify target biomolecules present in a sample in low abundance, relative to the abundance of non-target biomolecules in the sample.

It also is an object of the present invention to provide a biodetection method which can identify a target biomolecule in a sample mixture which comprises target and non-target biomolecules having a shared epitope, regardless of the abundance level of the target and non-target biomolecules.

It is a further object of the present invention to provide a biodetection method which is capable of enabling accurate measurement of a biomolecule in a sample, notwithstanding the abundance level of the target biomolecule.

It is an additional object of the present invention to provide a biodetection method which permits accurate measurement of DNA, mRNA or protein in a complex biological sample.

It is another object of the present invention to provide a biodetection method which eliminates artifactual signals produced by cross-reaction of the target probe with the non-target biomolecule.

It is yet another object of the present invention to provide a biodetection method which is capable of being used with any standard blotting technique, including the immunoblot (Western blot), Southern blot and Northern blot.

It is still another object of the present invention to provide a biodetection method which is capable of detecting biomolecules in complex mixtures including, for example, histological tissue sections, cell lysates and body fluids.

Additional objects, advantages and novel features of the invention will be set forth in part of the description and claims which follows, and in part will become apparent to those skilled in the art upon examination of the following specification or may be learned by practice of the invention.

These and other objects of the present invention are achieved by providing a biodetection method which improves biomolecule detection specificity by utilizing a combination of at least one target probe and a competitor molecule in order to identify a target biomolecule in a sample. In a second embodiment, the biodetection method utilizes two target probes in combination with a competitor molecule when the sample contains a target biomolecule and a non-target biomolecule having a shared epitope. A detectable signal is produced when the at least one target probe is in high concentration relative to the competitor molecule, thereby identifying the target biomolecule. If the competitor molecule is in high concentration in the vicinity of the at least one target probe, the a detectable signal is not produced.

### BRIEF DESCRIPTION OF THE DRAWINGS

The present invention will be better understood with reference to the appended drawing sheets, wherein:

FIG. 1A shows a standard immunoblot without detection of any target proteins before probing.

FIG. 1B shows the same standard immunoblot with detection of a target protein after probing.

FIG. 2A shows an example of an immunoblot failure due to a low abundance level of a target protein.

FIG. 2B shows an example of an immunoblot failure due to a shared epitope between a target protein and non-target protein.

FIG. 3 shows an immunoblot with the detection of a target protein in a sample using the biodetection method of the present invention, when the target protein has a low abundance level.

FIG. 4 shows an immunoblot with the detection of a target protein in a sample using the biodetection method of the present invention, when the target protein and a non-target protein have a shared epitope.

FIG. 5 shows a detection reaction utilizing an enzyme.

FIG. 6 shows a detection reaction utilizing “sticky-ended” DNA molecules.

FIG. 7 also shows a detection reaction utilizing “sticky-ended” DNA molecules.

### DETAILED DESCRIPTION

The present invention relates to a biodetection method which improves biomolecule detection specificity by utilizing a combination of at least one target probe and a competitor molecule in order to identify a target biomolecule in a sample. In a second embodiment, the biodetection method utilizes two target probes in combination with a competitor molecule when the sample contains a target biomolecule and a non-target biomolecule having a shared epitope. The method of the present invention will be described hereinafter using a standard immunoblot (Western blot). However, it is to be understood that the biodetection method of the present invention also can be applied to the detection of nucleic acids and/or other biological samples.

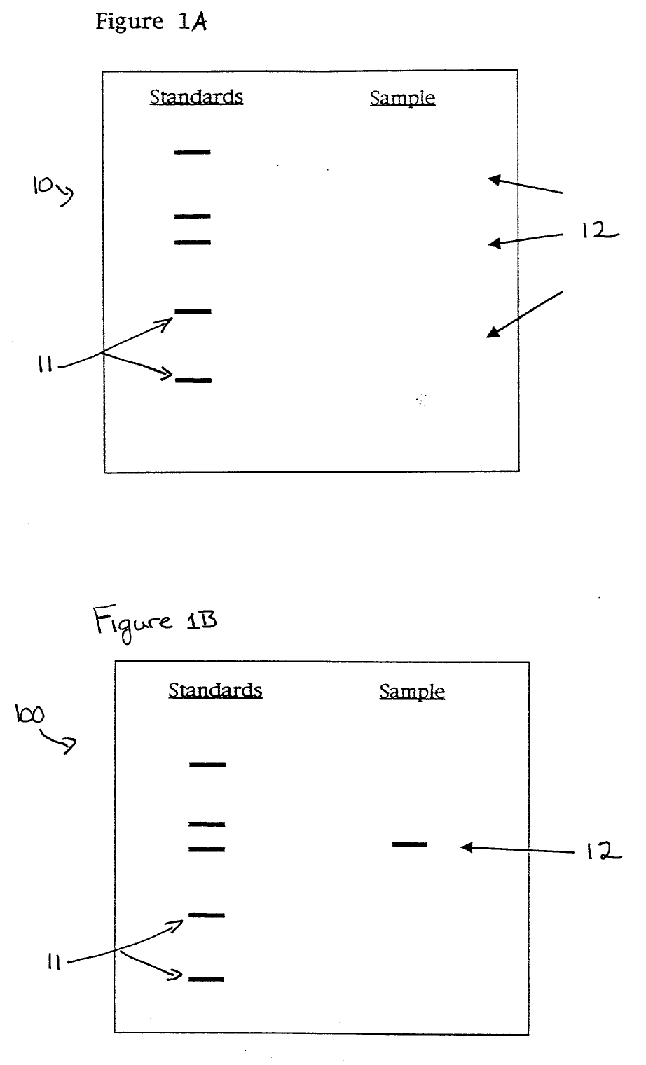

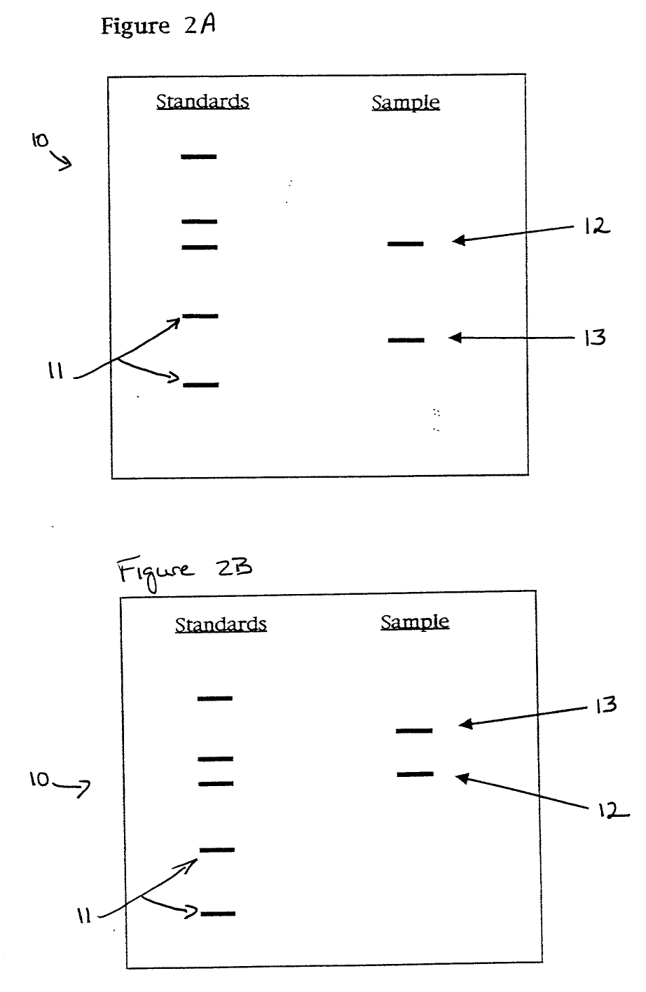

Referring to FIGS. 1A and 1B, before probing, a standard immunoblot 10 is shown having a plurality of standards 11 and undetected proteins 12. After probing the blot with a target antibody, a target protein is identified as shown in immunoblot 100. However, this standard probing technique will fail, as shown in FIG. 2A when the target protein 12 is in low abundance relative to one or more non-target proteins 13 in the sample. This failure is the result of interaction between the target antibody and the one or more non-target proteins. Such an interaction will generate a significant artifactual signal relative to the signal produced by the interaction of the target antibody with the target protein. Thus, the investigator will be not be able to ascertain which band on the blot represents the target molecule and which band is due to the target antibody cross-reaction with the non-target protein(s). Even a relatively weak interaction between the target antibody and a highly abundant, non-target protein(s) will generate a significant artifactual signal.

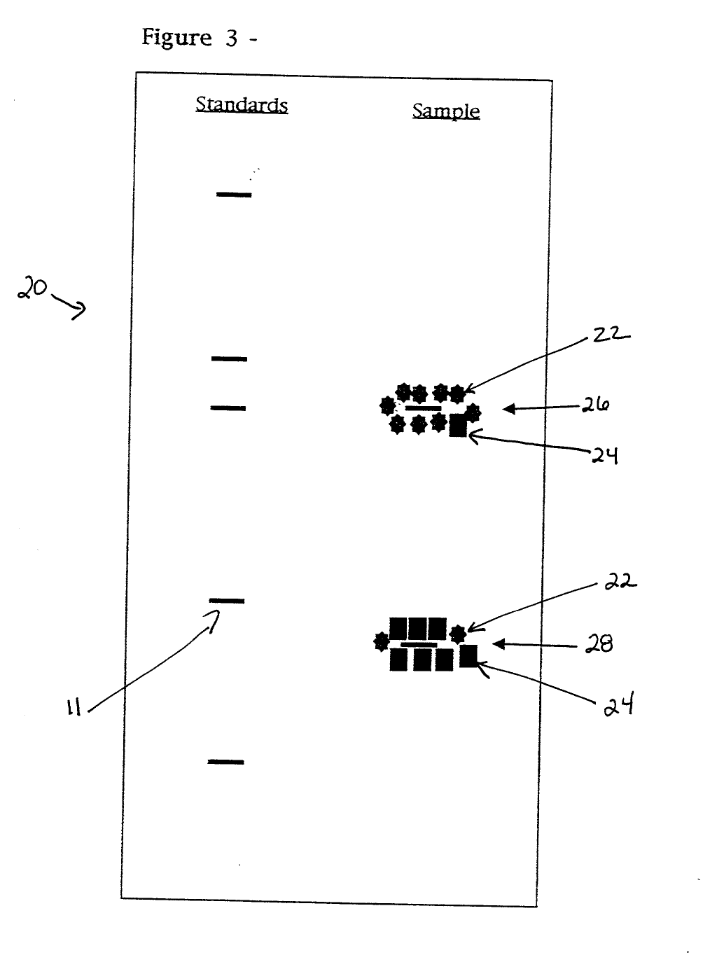

In order to overcome this failure, the biodetection method of the present invention utilizes one target antibody against the target protein along with a competitor molecule. As shown in FIG. 3, the inventive biodetection method generates a highly specific target protein signal. More specifically, a single target antibody (Tab) 22 that is specific for the target protein 26 is used to probe the immunoblot and specifically binds to the target protein in high concentration due to the strong affinity for this molecule. The target antibody also may bind to non-target proteins 28 that are present in the sample in high-abundance. That is, the target antibody binds non-specifically and thus binds in proportion to the abundance level of each non-target protein.

A competitor molecule is added to the immunoblot in high concentration, for example, at about 500× to about 1,500×, preferably about 1000×, the level of the target antibody. Solely for the purpose of illustrating the invention, the competitor molecule will hereinafter be described as a competitor antibody (Cab) 24. However, as will be obvious to those skilled in the art, any competitor molecule can be utilized in the present invention. The competitor antibody binds non-specifically to all of the proteins in the sample in proportion to the abundance level of each protein. Thus, the target protein has been bound by a high concentration of the target antibody due to the specific affinity of the target antibody for the target protein. Since it is present in low abundance in the sample, the target protein has been bound non-specifically by a low concentration of competitor antibody. The high-abundance, non-target protein(s) in the sample has been bound non-specifically by both the target antibody and competitor antibody. However, since the competitor antibody was added at high concentration, preferably at about 1000×concentration to the immunoblot, it will be bound to the non-target protein at 1000×relative to the target antibody.

In order to detect the target protein, a detection reaction can be initiated (as described more fully hereinafter) that produces a signal only when the target antibody is in high concentration relative to competitor antibody. As only the target antibody is in high concentration on the target protein, the specific signal is generated from the target protein, but not from the non-target protein in the sample. In this manner, the target protein, and only the target protein is detected, as shown in FIG. 3.

The standard probing technique also will fail when the target antibody specifically cross-reacts with a protein other than the target protein due to a shared epitope between the target protein and non-target protein, as shown in FIG. 2B. This type of failure can occur for target and non-target proteins of any abundance level. However, it is particularly challenging when the target protein is in low abundance and/or the non-target protein is in high abundance.

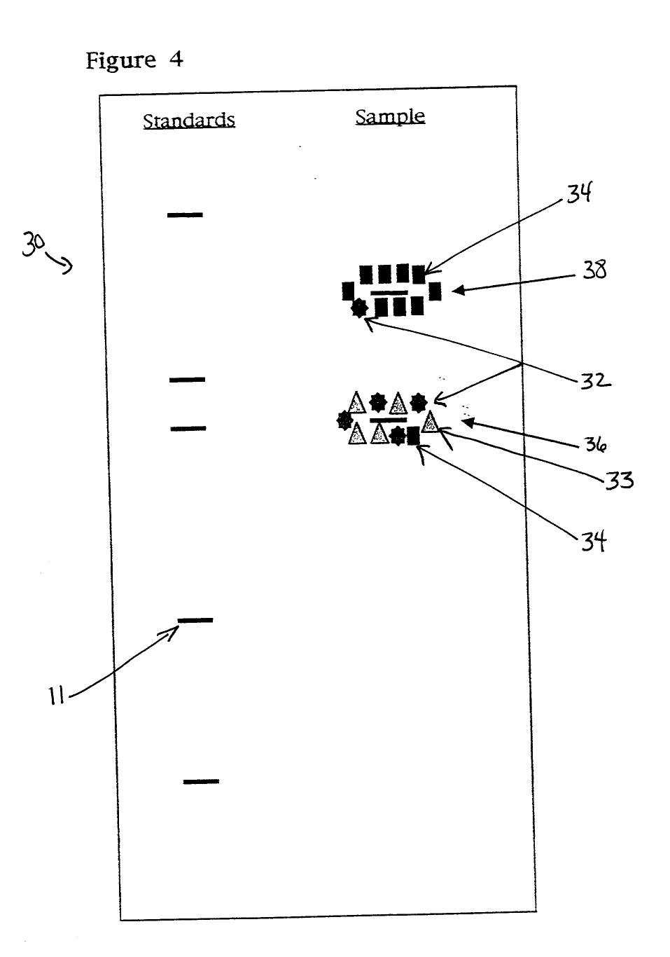

In order to overcome this failure, the biodetection method of the present invention utilizes two target antibodies against the target protein along with a competitor molecule. As shown in FIG. 4, the inventive biodetection method generates a highly specific target protein signal. More specifically, the two target antibodies 32 and 33 (Tab-1 and Tab-2) that are specific for the target molecule 36 are used to probe the immunoblot 30 and specifically bind to the target protein in high concentration due to their strong affinity for this molecule. It is to be understood that each of the two target antibodies can be separate monoclonal antibodies against the target protein and/or derived from a polyclonal antibody that recognizes two or more epitopes on the target protein. One or both of the two target antibodies also may bind to non-target proteins 38 of any abundance level that share a similar antibody epitope as one of the target antibodies.

A competitor molecule 34 is added to the immunoblot in high concentration, for example, at about 500× to about 1,500×, preferably about 1000×, the level of the target antibodies (Tab-1 and Tab-2). As noted above, solely for the purpose of illustration, the competitor molecule will hereinafter be described as a competitor antibody (Cab). However, as will be obvious to those skilled in the art, any competitor molecule can be utilized in the present invention. The competitor antibody binds non-specifically to all of the proteins in the sample in proportion to the abundance level of each protein. Thus, the target protein 36 has been bound by a high concentration of Tab-1 32 and Tab-2 33 due to the specific affinities of these antibodies for the target protein. When it is present in low abundance in the sample, the target protein also has been bound non-specifically by a low concentration of competitor antibody.

In order to detect the target protein, a detection reaction can be initiated (as described more fully hereinafter) between the two target antibodies (i.e. Tab-1 and Tab-2) and the competitor molecule (e.g. Cab) that produces a signal only when Tab-1 and Tab-2 antibodies are in close physical proximity to each other and in high concentration relative to competitor antibody. Since this such a situation only occurs on the target protein, a specific signal is generated from the target protein, but not from the non-target protein in the sample.

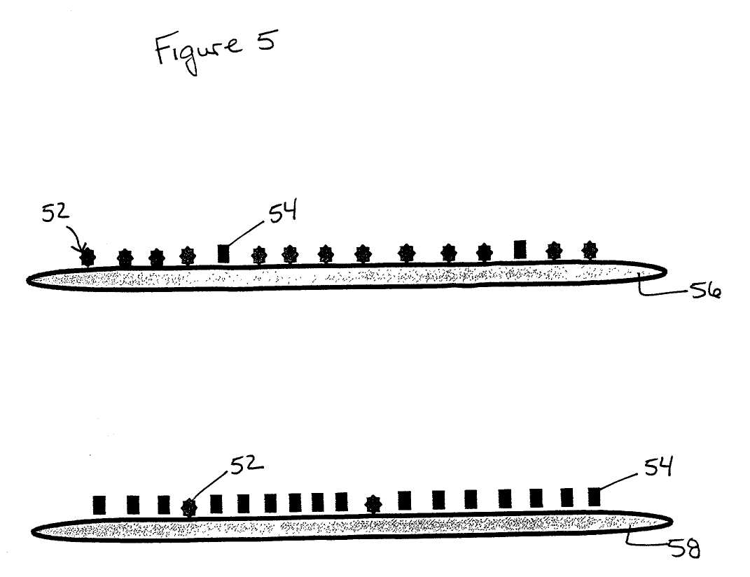

Several different detection reactions can be developed to generate the specific signal. An enzyme detection reaction can be produced using an enzyme that catalyzes a detectable reaction. The enzyme is attached to the target antibody (Tab) and an inhibitor of this enzyme is attached to the competitor molecule (Cab). The detection reaction is established only when the target antibody (Tab) is present on the target biomolecule in high concentration relative to the competitor molecule. Referring to FIG. 5, the concentration of the target antibody 52 (Tab) is significantly higher on the target protein 56 than the concentration of the competitor antibody 54 (Cab). Therefore, an enzyme attached to the target antibody is free to catalyze a detection reaction. On the non-target protein 58, the concentration of the competitor antibody 54 (Cab) is much higher than the target antibody 52 (Tab). Thus, when the competitor antibody is attached to an inhibitor of the enzyme, the detectable reaction is prevented from occurring. Any type of enzyme that catalyzes a detectable reaction is suitable for use in the present invention. Suitable enzymes include, for example, alkaline phosphatase, horseradish peroxidase and lactoperoxidase.

Another type of detection reaction suitable for use with the present invention is a fluorochrome label-produced detection reaction. In this detection reaction, which is particularly useful for the method to overcome the failure shown in FIG. 2B, each of the two target antibodies (Tab-1 and Tab-2) and the competitor antibody (Cab) are labeled with different fluorochromes. In this manner, a specific emission signal is produced only when the Tab-1 and Tab-2 are in close proximity to one another and the Cab is not present.

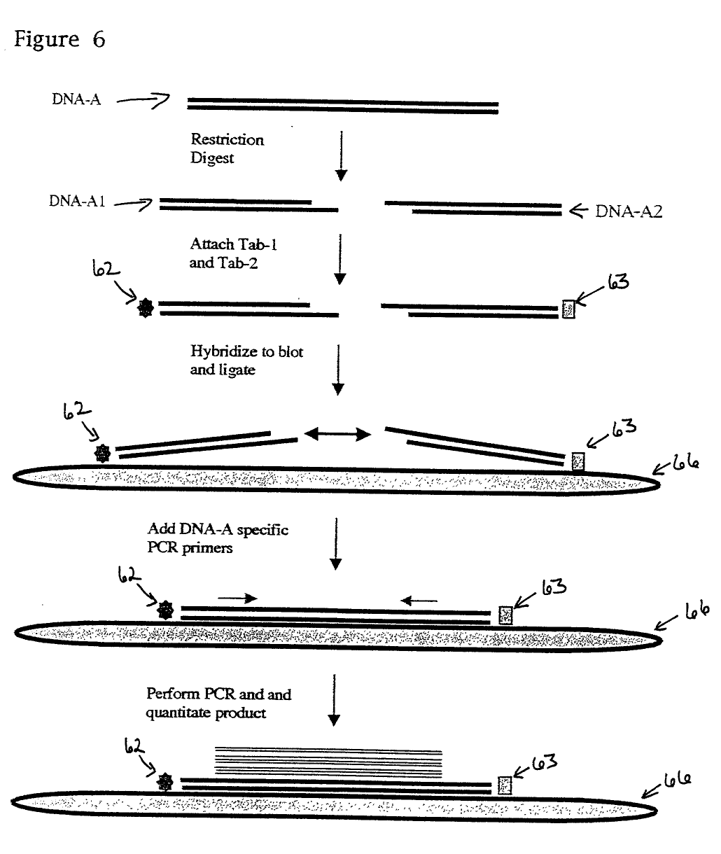

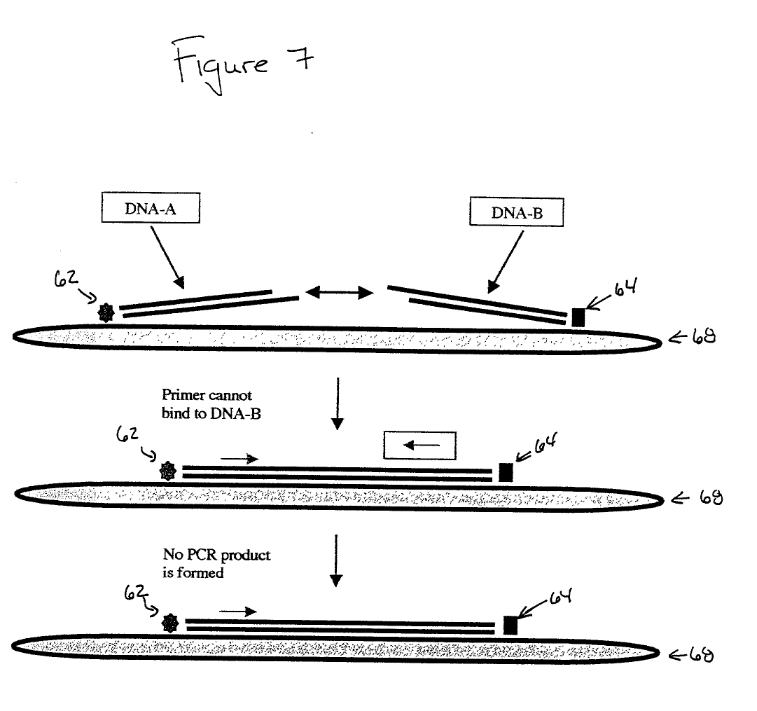

A different type of detection reaction suitable for use with the present invention, particularly with respect to the failure shown in FIG. 2B, utilizes “sticky-ended” DNA molecules. In this detection reaction, the “sticky-ended” DNA molecules are attached to the two target antibodies (Tab-1 and Tab-2) and the competitor antibody, as shown in FIGS. 6 and 7.

More specifically, as shown in FIG. 6, a DNA molecule of 50 bp in length (DNA-A) is cut in the middle with a restriction endonuclease, thus creating two DNA fragments (denoted DNA-A1, DNA-A2) that contain complementary “sticky-ends”. Tab-1 62 is attached to the “non-sticky end” of DNA-A1 and Tab-2 63 is attached to the “non-sticky end” of DNA-A2. In this manner, each “non-sticky-end” of the DNA fragment is attached to one of the target antibodies, thereby leaving each of the complementary “sticky-ends” free to interact in subsequent steps of the detection reaction. The competitor antibody 64 (Cab) is similarly attached to “sticky-ended” DNA fragments created by a restriction digest of a DNA molecule (DNA-B) that has a different nucleotide sequence from DNA-A except for a shared restriction site in the middle.

After hybridization of the two the target antibodies (Tab-1 and Tab-2) and the competitor antibody (Cab) to the blot, a ligation reaction is performed that attaches together the sticky-ends of DNA molecules that are located close together. If Tab-1 and Tab-2 are in close proximity and in high concentration, and if the Cab is not present in high concentration that is, on the target protein, then DNA-A will be recreated, as shown in FIG. 6. However, if Cab is present in high concentration, a new DNA molecule will be created representing either a hybrid of DNA-A and DNA-B or re-creating the original DNA-B molecule, as shown in FIG. 7.

A PCR reaction is performed on the blot using PCR primers specific for DNA-A. This reaction is performed similar to in-situ PCR of a histologic tissue section; that is, the PCR product remains spatially localized in the region where it is produced. Thus, in this manner, a specific signal is generated from the target molecule 66 on the blot only where the DNA-A molecule is present (i.e., the target protein), but is not produced where a DNA-A/B hybrid has been made or where the DNA-B molecule is re-created (i.e., the non-target protein 68). The level of the PCR product on the blot is quantitated, thus providing a highly specific measurement of the target protein, as shown in FIG. 6. Due to the ability of PCR to greatly amplify DNA, this particular method also provides enhanced detection sensitivity of the target protein.

While particular embodiments of the invention have been described, it will be understood, of course, that the invention is not limited thereto, and that many obvious modifications and variations can be made, and that such modifications and variations are intended to fall within the scope of the appended claims.

What is claimed is:

1. A method for the detection of a target biomolecule in a sample comprising:
(a) adding to said sample a target molecule that has a strong affinity to said target biomolecule such that it will specifically bind to said target biomolecule in high concentration;
(b) adding to said sample a competitor molecule at a level of from about 500 to about 1,500 times the level of said target molecule, said competitor molecule being capable of binding non-specifically to all biomolecules in said sample in proportion to the abundance level of each biomolecule, and
(c) detecting said target biomolecule in said sample by initiating a detection reaction that produces a signal only when said target molecule is in high concentration on said target biomolecule, relative to said concentration of said competitor molecule on said target biomolecule.

2. The method in accordance with claim 1, wherein said target biomolecule is a target protein, said target molecule is a target antibody, and said competitor molecule is a competitor antibody.

3. The method in accordance with claim 1, wherein said detection reaction is an enzyme detection method.

4. The method in accordance with claim 2, wherein said detection reaction is an enzyme detection method comprises attaching to said target antibody an enzyme that catalyzes a detectable reaction and attaching to said competitor antibody an inhibitor of said enzyme.

5. The method in accordance with claim 4, wherein said enzyme is selected from the group consisting of alkaline phosphatase, horseradish peroxidase and lactoperoxidase.

6. The method in accordance with claim 5, wherein said enzyme is alkaline phosphatase.

7. The method in accordance with claim 1, wherein said detection reaction is a fluorochrome label-produced detection reaction.

8. The method in accordance with claim 2, wherein said detection reaction is a fluorochrome label-produced detection reaction comprising labeling said target antibody with a first fluorochrome label and said competitor with a second fluorochrome label.

9. A method for the detection of a target biomolecule in a sample comprising:
(a) adding to said sample a first target molecule and a second target molecule, both said first target molecule and said second target molecule having a strong affinity to said target biomolecule such that both said first target molecule and said second target molecule will specifically bind to said target biomolecule in high concentration;
(b) adding to said sample a competitor molecule at a level of from about 500 to about 1,500 times the combined level of said first target molecule and said second target molecule, said competitor molecule being capable of binding non-specifically to all biomolecules in said sample in proportion to the abundance level of each biomolecule, and
(c) detecting said target biomolecule in said sample by initiating a detection reaction that produces a signal only when both said first target molecule and said second target molecule are in close proximity to each other and in high concentration on said target biomolecule, relative to said concentration of said competitor molecule on said target biomolecule.

10. The method in accordance with claim 9, wherein said target biomolecule is a target protein, said first target molecule is a first target antibody, said second target molecule is a second target antibody, and said competitor molecule is a competitor antibody.

11. The method in accordance with claim 9, wherein said detection reaction is an enzyme detection method.

12. The method in accordance with claim 11, wherein said detection reaction is an enzyme detection method comprises attaching to both said first target antibody and said second target antibody an enzyme that catalyzes a detectable reaction and attaching to said competitor antibody an inhibitor of said enzyme.

13. The method in accordance with claim 12, wherein said enzyme is selected from the group consisting of alkaline phosphatase, horseradish peroxidase and lactoperoxidase.

14. The method in accordance with claim 13, wherein said enzyme is alkaline phosphatase.

15. The method in accordance with claim 9, wherein said detection reaction is a fluorochrome label-produced detection reaction.

16. The method in accordance with claim 10, wherein said detection reaction is a fluorochrome label-produced detection reaction comprising labeling said first target antibody with a first fluorochrome label, said second target antibody with a second fluorochrome label and said competitor with a third fluorochrome label.

17. The method in accordance with claim 16, wherein in said detection reaction, a specific emission signal is produced only when said first target antibody and said second target antibody are in close proximity to one another and when said competitor antibody is not present.

18. The method in accordance with claim 9, wherein said detection reaction is a DNA-fragmentation detection reaction.

19. The method in accordance with claim 10, wherein said detection reaction is a DNA-fragmentation detection reaction comprising:
(a) fragmenting a first DNA molecule into a first DNA fragment and a second DNA fragment, each having a complementary sticky end and a non-sticky end;
(b) attaching said first target antibody to said non-sticky end of said first DNA fragment and said second target antibody to said non-sticky end of said second DNA fragment, thereby leaving each of complementary sticky ends free;
(c) fragmenting a second DNA molecule having a different nucleotide sequence than said first DNA molecule into one or more fragments each having a sticky end and a non-sticky end and attaching the competitor antibody to said non-sticky end;
(d) hybridizing all of said first and second target antibodies and said competitor antibody to said sample;
(e) performing a ligation reaction that attaches together the sticky ends of said DNA fragments that are in close proximity in such a manner that the first DNA molecule will be re-created when said first target antibody and said second target antibody are in close proximity and in high concentration and when said competitor antibody is not present in high concentration, and
(f) performing a PCR reaction using PCR primers specific for the first DNA molecule, thereby generating a specific signal from said target molecule only when said first DNA molecule is present.
